(19) United States
(12) Patent Application Publication
(10) Pub. No.: US 2022/0167871 A1
(43) Pub. Date: Jun. 2, 2022

(54) INFORMATION GENERATING APPARATUS, INFORMATION GENERATING METHOD, AND NON-TRANSITORY COMPUTER-READABLE MEDIUM

(71) Applicant: NIHON KOHDEN CORPORATION, Tokyo (JP)

(72) Inventors: Takayuki SUGIYAMA, Tokorozawa-shi (JP); Hironori KOBAYASHI, Tokorozawa-shi (JP); Kazuri HIGASHI, Tokorozawa-shi (JP); Ryota AUCHI, Tokorozawa-shi (JP)

(21) Appl. No.: 17/529,614

(22) Filed: Nov. 18, 2021

(30) Foreign Application Priority Data
Nov. 27, 2020 (JP) 2020-197106

(51) Int. Cl. A61B 5/08; A61B 5/087; A61B 5/00

(52) U.S. Cl. CPC A61B 5/0816; A61B 5/742; A61B 5/7235; A61B 5/087

(57) ABSTRACT

An information generating apparatus is provided with an acquirer and a controller. The acquirer is configured to acquire respiratory waveform data about respiratory pressure of a subject. The controller is configured to compare the respiratory waveform data acquired by the acquirer with preset respiratory reference waveform data, so as to generate respiratory depth information representing respiratory depth of the respiratory waveform data with respect to the respiratory reference waveform data.

### CROSS-REFERENCE TO RELATED APPLICATIONS

The present application claims priority from Japanese Patent Application No. 2020-197106, filed Nov. 27, 2020, the entire content of which is incorporated herein by reference.

### TECHNICAL FIELD

The presently disclosed subject matter relates to an information generating apparatus, an information generating method, and a non-transitory computer-readable medium on which a computer program for making the apparatus execute the method has been recorded.

### BACKGROUND ART

JP-A-2018-201725 discloses a physiological information processing apparatus that analyzes a measurement result of intranasal respiratory pressure in real time and displays the analyzed result on a display.

A state of respiration of a subject is generally judged based on frequency of the respiration and depth of the respiration. The frequency of the respiration can be objectively judged from a rate of the respiratory. On the other hand, the depth of the respiration is subjectively judged by a medical worker who visually checks a waveform of the respiration. For this reason, it is difficult for the medical worker to judge the depth of the respiration objectively. Since the physiological information processing apparatus according to JP-A-2018-201725 can calculate the respiratory rate of the subject, the medical worker can objectively grasp the respiratory frequency of the subject by use of such a physiological information processing apparatus, but cannot objectively grasp the respiratory depth of the subject. In this respect, there is still room for improvement in the background-art physiological data processing apparatus.

### SUMMARY

An object of the presently disclosed subject matter is to provide an information generating apparatus that can objectively grasp respiratory depth of a subject, an information generating method, and a non-transitory computer-readable medium on which a computer program for making the apparatus execute the method has been recorded.

An information generating apparatus according to a first aspect for achieving the aforementioned object may include:
an acquirer that is configured to acquire respiratory waveform data about respiratory pressure of a subject; and
a controller that is configured to compare the respiratory waveform data acquired by the acquirer with preset respiratory reference waveform data, so as to generate respiratory depth information representing respiratory depth of the respiratory waveform data with respect to the respiratory reference waveform data.

In addition, an information generating method according to a second aspect for achieving the aforementioned object may be attained by making an information generating apparatus execute:
a step of acquiring respiratory waveform data about respiratory pressure of a subject; and
a step of comparing the acquired respiratory waveform data with preset respiratory reference waveform data, so as to generate respiratory depth information representing respiratory depth of the respiratory waveform data with respect to the respiratory reference waveform data.

In addition, a computer program according to a third aspect for achieving the aforementioned object may be attained by making a computer realize:
a function of acquiring respiratory waveform data about respiratory pressure of a subject; and
a function of comparing the acquired respiratory waveform data with preset respiratory reference waveform data, so as to generate respiratory depth information representing respiratory depth of the respiratory waveform data with respect to the respiratory reference waveform data.

Moreover, the aforementioned computer program has been recorded on a non-transitory computer-readable medium according to a fourth aspect for achieving the aforementioned object.

According to the information generating apparatus, the information generating method, the computer program and the non-transitory computer-readable medium according to the aforementioned configurations, the respiratory reference waveform data serving as a reference for determining the respiratory depth of the subject and the respiratory waveform data acquired from the subject are compared so that the respiratory depth information is generated. Therefore, the respiratory depth information is information that objectively represents the respiratory depth of the subject. Accordingly, for example, a medical worker who visually checks a display screen based on the respiratory depth information does not have to judge the respiratory depth based on the medical worker's personal experience or the like as in the background art but can judge the respiratory depth based on the respiratory depth information that is an objective index.

### ADVANTAGEOUS EFFECTS OF INVENTION

According to the presently disclosed subject matter, it is possible to provide an information generating apparatus that can objectively grasp respiratory depth of a subject, an information generating method, and a non-transitory computer-readable medium on which a computer program for making the apparatus execute the method has been recorded.

### BRIEF DESCRIPTION OF DRAWINGS

FIG. 1 is a functional block diagram of an information generating apparatus according to an embodiment of the presently disclosed subject matter.

FIG. 2 is a flow chart of an information generating method according to the embodiment of the presently disclosed subject matter.

FIG. 3 is an example of a physiological waveform including a respiratory waveform of a subject.

FIG. 4 is an example of a respiratory reference waveform of the subject.

FIG. 5 is an example of a display screen according to the embodiment of the presently disclosed subject matter.

FIG. 6 is an example of the respiratory waveform of the subject.

FIG. 7 is another example of the display screen according to the embodiment of the presently disclosed subject matter.

FIG. 8 is an example of a display screen according to another embodiment of the presently disclosed subject matter.

### DESCRIPTION OF EMBODIMENTS

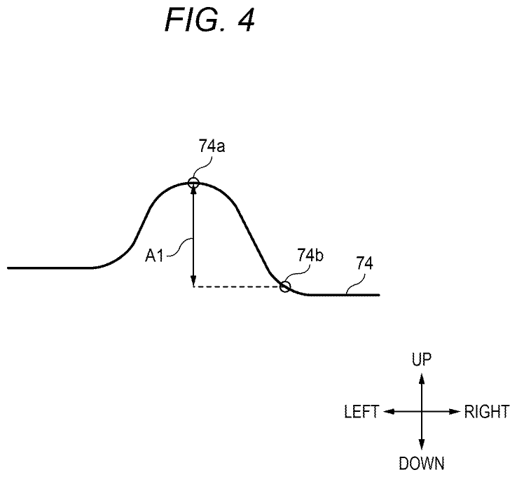

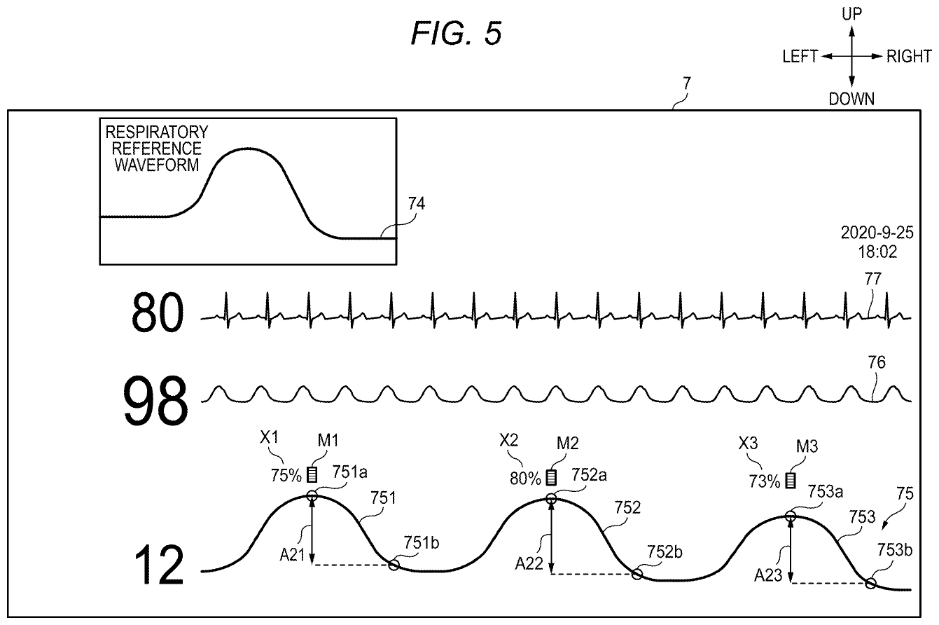

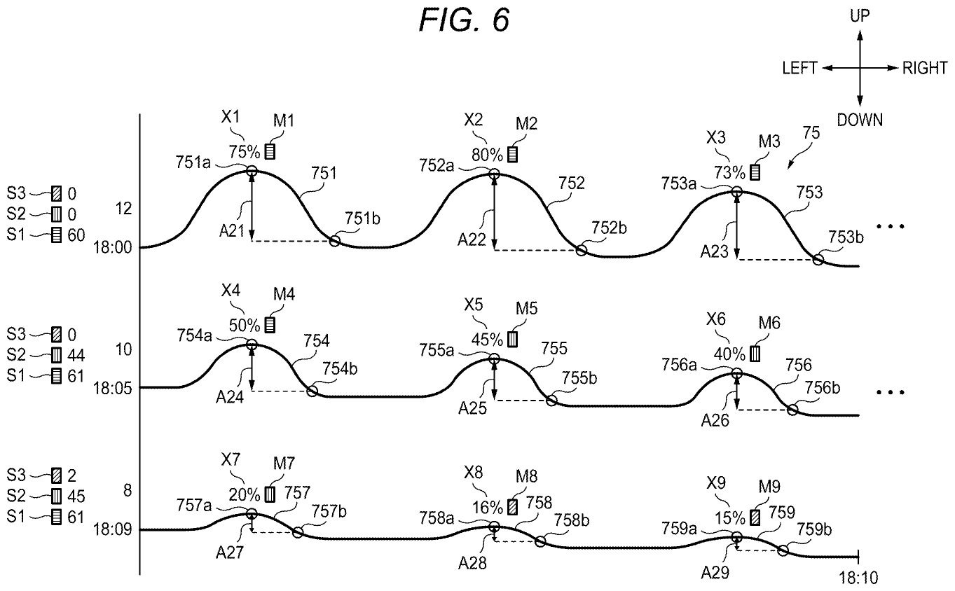

Embodiments of the presently disclosed subject matter will be described below by way of example with reference to the drawings. Incidentally, in description of each of the embodiments, “left-right direction” and “up-down direction” will be referred to appropriately for convenience of explanation. These directions are relative directions set in waveforms respectively illustrated in FIG. 4 and FIG. 6, or in a display 7 illustrated in each of FIG. 5 and FIGS. 7 and 8.

### First Embodiment

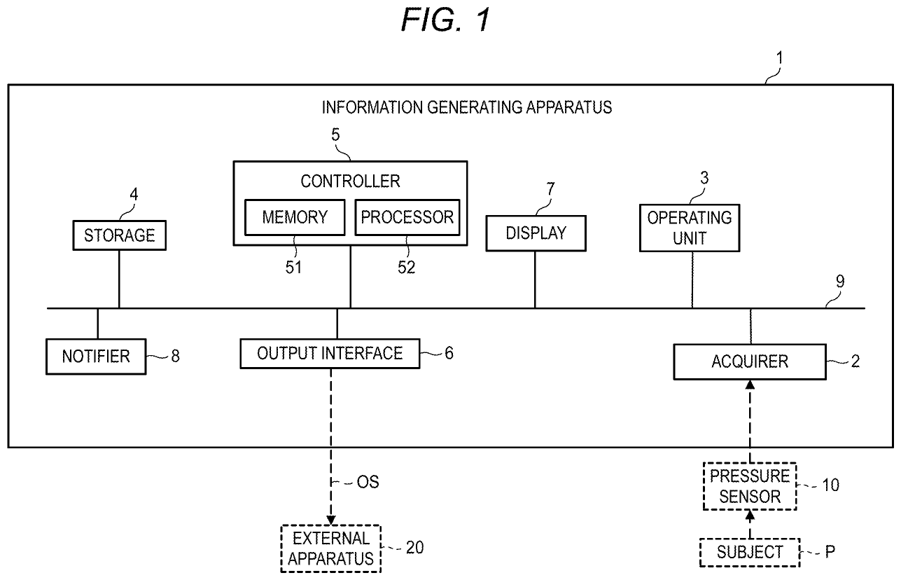

FIG. 1 is a functional block diagram of an information generating apparatus 1 according to an embodiment of the presently disclosed subject matter. The information generating apparatus 1 is, for example, a bedside monitor. As illustrated in FIG. 1, the information generating apparatus 1 is provided with an acquirer 2, an operating unit 3, a storage 4, a controller 5, an output interface 6, the display 7, and a notifier 8, which are communicably connected to one another through a bus 9.

The acquirer 2 is configured to acquire physiological information including respiratory waveform data about respiratory pressure of a subject P from the subject P. The respiratory waveform data are based on a physiological signal corresponding to respired air from at least one of the mouth and nose of the subject P detected by a pressure sensor 10. The physiological information acquired by the acquirer 2 may include ECG waveform data, transcutaneous arterial blood oxygen saturation data, and the like, detected by various sensors. The physiological information acquired by the acquirer 2 is transmitted to the storage 4 or the controller 5.

The operating unit 3 is configured to accept an input operation made by a person (e.g. a medical worker) operating the information generating apparatus 1 and to generate an instruction signal corresponding to the input operation. The operating unit 3 is, for example, a touch panel arranged to be superimposed on the display 7, an operation button attached to a housing of the information generating apparatus 1, and the like. The operating unit 3 accepts any of various input operations etc., generates an instruction signal corresponding to the input operation, and transmits the generated instruction signal to the controller 5.

The storage 4 is, for example, a storage device such as an HDD (Hard Disk Drive) or an SSD (Solid State Drive). The storage 4 stores the physiological information that has been acquired by the acquirer 2, information that has been inputted through the operating unit 3, information that has been generated by the controller 5, etc.

The controller 5 is provided with a memory 51 and a processor 52. The memory 51 is constituted by, for example, an ROM (Read Only Memory) in which various programs, etc. have been stored, a RAM (Random Access Memory) that has a plurality of work areas where various programs etc. executed by the processor 52 are stored, etc. The processor 52 which is, for example, a CPU (Central Processing Unit) is configured to expand a program designated from the various programs incorporated in the ROM onto the RAM and execute various processings in cooperation with the RAM.

The controller 5 is configured to generate respiratory depth information, which is information about depth of respiration. The generated respiratory depth information can be transmitted to the storage 4. Incidentally, the respiratory depth information includes classification information according to a classified state of the respiratory depth of the subject P, and numerical value information expressing the state of the respiratory depth of the subject P by a numerical value.

The controller 5 is configured to determine, based on the respiratory depth information, whether it is necessary to send notification of the respiratory depth state of the subject P or not. Incidentally, the controller 5 may be configured to determine whether it is necessary to send notification of the respiratory depth state of the subject P or not using not only the respiratory depth information but also, for example, aggregate information etc. which will be described later. After determining that it is necessary to send notification of the respiratory depth state of the subject P, the controller 5 is configured to generate a notification signal for notifying the medical worker or the like of the respiratory depth state of the subject P. The generated notification signal is transmitted to an external apparatus 20 through the notifier 8 or the output interface 6.

The controller 5 is configured to generate the aggregate information in which pieces of such respiratory depth information in a predetermined time are counted up. A length of the predetermined time can be desirably set by the medical worker or the like. The predetermined time is, for example, 10 minutes. The generated aggregate information can be transmitted to the storage 4.

The controller 5 is configured to generate display data for displaying the respiratory waveform data and the respiratory depth information on the display 7 or a display provided in the external apparatus 20. The generated display data are transmitted to the output interface 6 or the display 7.

The output interface 6 is configured to output an output signal OS corresponding to the information which has been transmitted to the output interface 6. The output signal OS can be transmitted to the external apparatus 20. The output interface 6 can be provided with a circuit if necessary. The circuit converts output data into the output signal OS that can be processed by the external apparatus 20.

The external apparatus 20 is configured to notify the medical worker or the like of various information by at least one of visible notification, audible notification, and tactile notification. The external apparatus 20 is, for example, a tablet terminal, a smartphone, or the like.

The display 7 is configured to display a display screen corresponding to the display data that have been received from the controller 5. The display 7 is, for example, a touch screen type display such as a liquid crystal display or an organic EL display, or the like. In addition to the respiratory waveform data and the respiratory depth information, other information may be also displayed on the display 7. The other information includes, for example, information about transcutaneous arterial blood oxygen saturation, a heart rate, an ECG, etc.

The notifier 8 is configured to send notification of the respiratory depth state of the subject P based on the notification signal which has been received from the controller 5. Incidentally, a notification mode made by the notifier 8 is similar to or the same as a notification mode made by the external apparatus 20.

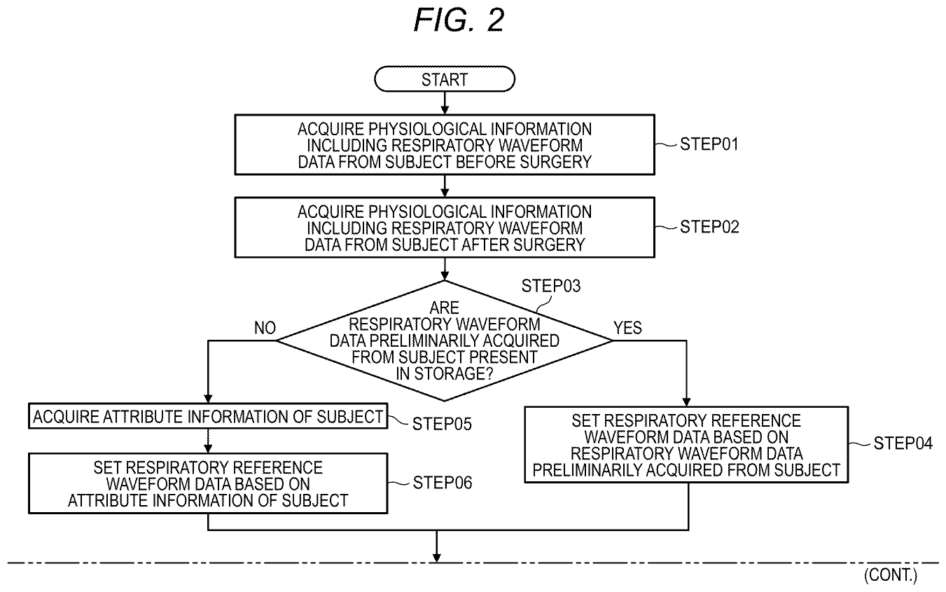

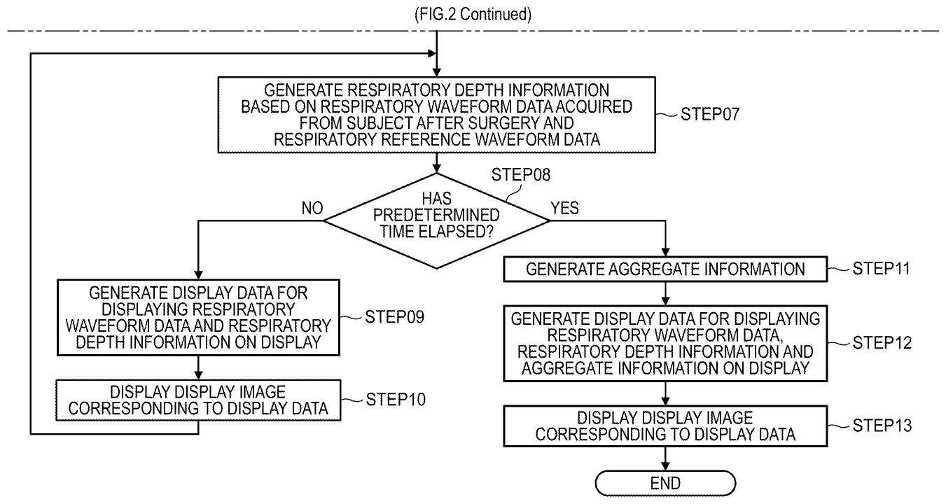

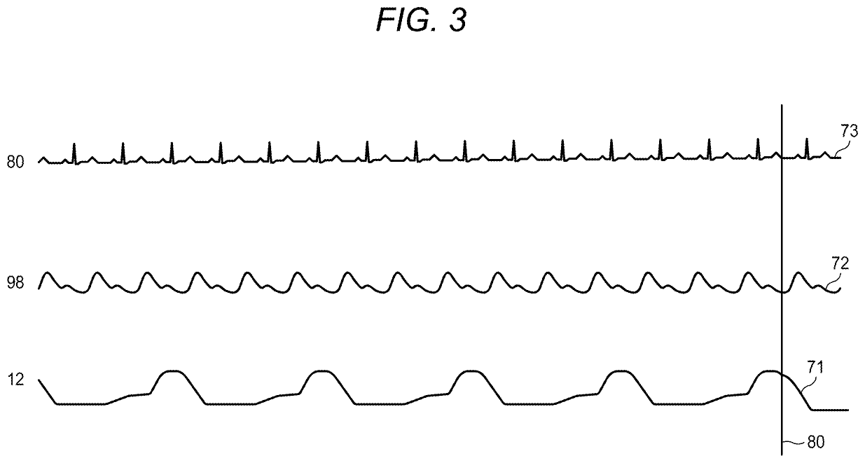

Next, an information generating method used in the presently disclosed embodiment will be described with reference to FIGS. 2 to 7. FIG. 2 is a flow chart of the information generating method according to the presently disclosed embodiment. As described in FIG. 2, the acquirer 2 acquires physiological information from a subject P before surgery (STEP01). When, for example, a medical worker performs an operation on the operating unit 3 to acquire the physiological information from the subject P, an instruction signal corresponding to the operation is transmitted from the operating unit 3 to the controller 5. Based on the instruction signal, the controller 5 controls the acquirer 2 to acquire respiratory waveform data from the subject P. In the presently disclosed embodiment, when a physiological waveform based on the physiological information acquired from the subject P in the STEP01 is displayed on the display 7, the physiological waveform illustrated in FIG. 3 is displayed on the display 7. The physiological waveform includes a respiratory waveform 71, a transcutaneous arterial blood oxygen saturation waveform 72, and an ECG waveform 73. Incidentally, in the example illustrated in FIG. 3, a respiratory rate is 12, a transcutaneous arterial blood oxygen saturation value is 98, and a heart rate is 80. In addition, in FIG. 3, a reference sign “80” indicates that the respiratory waveform 71, the transcutaneous arterial blood oxygen saturation waveform 72, and the ECG waveform 73 are in the same phase. After acquiring the physiological information from the subject P, the acquirer 2 transmits the acquired physiological information to the storage 4.

In the presently disclosed embodiment, the subject P undergoes surgery after the STEP01, and an anesthetic is administered to the subject P during the surgery. When the anesthetic administered during the surgery still remains in the body of the subject P, there is a possibility that respiration of the subject P after the surgery is shallower than respiration of the subject P on a normal occasion due to the remaining anesthetic. Especially during sleeping, there is a possibility that the subject P breathes more shallowly due to the effect of the remaining anesthetic. Therefore, as described in FIG. 2, the physiological information is acquired from the subject P after the surgery by the acquirer 2 when much time does not elapse after completion of the surgery (STEP02). A process of acquiring the physiological information in the STEP02 is similar to or the same as a process of acquiring the physiological information in the STEP01. When a respiratory waveform 75 (an example of a second respiratory waveform) based on the respiratory waveform data acquired in the STEP02 is displayed on the display 7, for example, a waveform illustrated in FIG. 5 is displayed on the display 7. Incidentally, the respiratory waveform data include expiratory waveform data of the subject P (e.g. data about an amount of expired air of the subject P and detection time of the expiration) and inspiratory waveform data of the subject P (e.g. data about an amount of inspired air of the subject P and detection time of the inspiration).

As described in FIG. 2, when the physiological information is acquired from the subject P after the surgery, the controller 5 determines whether respiratory waveform data that have been preliminarily acquired from the subject P are present in the storage 4 or not (STEP03). In the presently disclosed embodiment, since the respiratory waveform data that have been preliminarily acquired from the subject P are present in the storage 4 (YES in the STEP03), the controller 5 sets respiratory reference waveform data based on the preliminarily acquired respiratory waveform data (STEP04). Incidentally, the respiratory reference waveform data may be automatically set by the controller 5, or may be set by an input operation to the operating unit 3 by the medical worker. Moreover, since an ordinary respiratory state (respiratory depth) of the subject is normally known before the surgery, the controller 5 sets respiratory waveform data as a reference (respiratory reference waveform data) from the respiratory waveform data which have been acquired from the subject P before the surgery. Furthermore, when a respiratory reference waveform 74 (an example of a first respiratory waveform) based on the respiratory reference waveform data set in the STEP04 is displayed on the display 7, a waveform illustrated in FIG. 4 is displayed on the display 7. The respiratory reference waveform 74 is a waveform indicating a standard respiratory pressure level of the subject P.

On the other hand, as described in FIG. 2, when the respiratory waveform data which have been preliminarily acquired from the subject P are absent from the storage 4 (NO in the STEP03), the controller 5 acquires attribute information of the subject P that has been stored in the storage 4 (STEP05). Incidentally, the attribute information is, for example, age information, gender information, chronic disease information, pre-existing disease information, etc.

After acquiring the attribute information of the subject P, the controller 5 sets respiratory reference waveform data based on the acquired attribute information (STEP06). In this case, for example, the controller 5 sets the respiratory reference waveform data based on statistical respiratory waveform data which have been acquired from other persons having attribute information the same as or close to the attribute information of the subject P. The other persons are, for example, subjects whose ages are close to that of the subject P, subjects who have the same chronic disease as the subject P, etc.

After setting the respiratory reference waveform data, the controller 5 generates respiratory depth information based on the respiratory waveform data acquired from the subject P after the surgery and the respiratory reference waveform data (STEP07).

Here, processing performed by the controller 5 in the STEP07 will be described in detail using FIG. 4 and FIG. 5. As illustrated in FIG. 4, the controller 5 determines a first amplitude A1 (an example of a first value) representing respiratory pressure based on the respiratory reference waveform data, based on inspiratory peak pressure 74a and expiratory peak pressure 74b in the respiratory reference waveform 74. The first amplitude A1 is a difference between a height of the inspiratory peak pressure 74a and a height of the expiratory peak pressure 74b.

As illustrated in FIG. 5, the respiratory waveform 75 includes a plurality of unit respiratory waveforms 751 to 753. Each of the unit respiratory waveforms is a respiratory waveform corresponding to a breath taken by the subject P. Based on inspiratory peak pressures 751a to 753a and expiratory peak pressures 751b to 753b in the unit respiratory waveforms 751 to 753, the controller 5 respectively determines second amplitudes A21 to A23 (an example of second values) representing respiratory pressures based on the respiratory waveform data.

The controller 5 respectively calculates relative values X1 to X3 (an example of numerical value information) indicating relative magnitudes of the second amplitudes A21 to A23 to the first amplitude A1. For example, the controller 5 calculates the relative value X1 based on the following equation (1).

X1=A21/A1×100 (%) (1)

In the presently disclosed embodiment, the magnitude of the second amplitude A21 is ¾ of the magnitude of the first amplitude A. Therefore, the relative value X1 is 75%.

The relative values X2 to X3 are also calculated in a manner similar to or the same as the relative value X1. In other words, the controller 5 compares the second amplitudes A21 to A23 of the respiratory waveform 75 included in the respiratory waveform data acquired by the acquirer 2 with the first amplitude A1 of the respiratory reference waveform 74 included in the respiratory reference waveform data, so as to generate the relative values X1 to X3 indicating the relative magnitudes of the second amplitudes A21 to A23 to the first amplitude A1. Incidentally, the relative values X1 to X3 are generated whenever the unit respiratory waveforms 751 to 753 are acquired.

The controller 5 classifies the respiratory depth state of the subject P based on the generated relative values X1 to X3 and classification criteria information that has been stored in the storage 4. The classification criteria information is information for setting criteria used for classifying the respiratory depth state. For example, the respiratory depth state can be classified into one of three state categories, that is, “normal, cautionary, and dangerous” states. In this case, the classification criteria information is, for example, thresholds etc. for defining boundaries (ranges) among the three categories. In the presently disclosed embodiment, the controller 5 classifies the respiratory depth state of the subject P as “dangerous” when the relative value is not more than 19%, classifies the respiratory depth state as “cautionary” when the relative value is not less than 20% and not more than 49%, and classifies the respiratory depth state as “normal” when the relative value is not less than 50%.

In the state illustrated in FIG. 5, all the relative values X1 to X3 are not less than 50%. Accordingly, the controller 5 classifies the respiratory depth state of the subject P corresponding to each of the unit respiratory waveforms 751 to 753 as “normal”. After classifying the respiratory depth state of the subject P corresponding to the unit respiratory waveform 751 to 753, the controller 5 generates classification information according to the classified respiratory depth state of the subject P. In this manner, the controller 5 generates respiratory depth information including the numerical value information and the classification information.

Return to FIG. 2. Processing in and after STEP08 will be described. In the STEP08, the controller 5 determines whether a predetermined time has elapsed or not since the acquirer 2 started acquiring the post-surgery respiratory waveform data from the subject P. Incidentally, in the presently disclosed embodiment, description will be made on the assumption that the predetermined time is 10 minutes. In a case where, for example, a time instant at which the acquirer 2 started acquiring the post-surgery respiratory waveform data from the subject P is 18:00 and a current time instant is 18:02, the controller 5 determines that the predetermined time (10 minutes) has not elapsed since 18:00 (NO in the STEP08). In this case, the controller 5 generates display data for displaying the respiratory waveform data and the respiratory depth information on the display 7 or on the display provided in the external apparatus 20 (STEP09).

The controller 5 is configured to generate either first display data or second display data as the display data. The first display data have inspiratory pressure higher than expiratory pressure in the respiratory waveform 75. The second display data have the expiratory pressure higher than the inspiratory pressure in the respiratory waveform 75. When, for example, the medical worker performs an operation on the operating unit 3 to make the controller 5 generate the first display data, the operating unit 3 generates an instruction signal corresponding to the input operation, and the controller 5 generates the first display data based on the instruction signal. In the presently disclosed embodiment, the inspiratory pressure is higher than the expiratory pressure in the respiratory waveform 75, as illustrated in FIG. 5. Therefore, the controller 5 generates the first display data. The generated first display data are transmitted to the output interface 6 or the display 7.

After generating the display data, the controller 5 controls the display 7 or the display provided in the external apparatus 20, so as to display a display screen corresponding to the generated display data (STEP10). In the presently disclosed embodiment, the controller 5 transmits the first display data to the display 7. When the display 7 receives the first display data from the controller 5, the display screen corresponding to the received first display data is displayed on the display 7. After the STEP10 is executed, the processing returns to the STEP07.

The display screen displayed on the display 7 at the time instant 18:02 will be described here with reference to FIG. 5. The display screen illustrated in FIG. 5 is displayed on the display 7 at the time instant 18:02. As illustrated in FIG. 5, the respiratory reference waveform 74, the respiratory waveform 75, a transcutaneous arterial blood oxygen saturation waveform 76, and an ECG waveform 77 are displayed on the display 7. The respiratory reference waveform 74, the respiratory waveform 75, the transcutaneous arterial blood oxygen saturation waveform 76 and the ECG waveform 77 are displayed side by side in the up-down direction. These waveforms are arranged side by side in the following order from the top: the respiratory reference waveform 74, the ECG waveform 77, the transcutaneous arterial blood oxygen saturation waveform 76, and the respiratory waveform 75. That is, the respiratory reference waveform 74 is displayed above the respiratory waveform 75, the transcutaneous arterial blood oxygen saturation waveform 76 and the ECG waveform 77, and the respiratory waveform 75 is displayed under the respiratory reference waveform 74, the transcutaneous arterial blood oxygen saturation waveform 76 and the ECG waveform 77. A length of the respiratory reference waveform in the left-right direction is about ⅖ of a length of the respiratory waveform in the left-right direction. A respiratory rate, a transcutaneous arterial blood oxygen saturation value and a heart rate are displayed respectively in the vicinities of left sides of the respiratory waveform 75, the transcutaneous arterial blood oxygen saturation waveform 76 and the ECG waveform 77. Incidentally, it is a matter of course that the display positions, etc. of the respiratory reference waveform 74, the respiratory waveform 75, the transcutaneous arterial blood oxygen saturation waveform 76 and the ECG waveform 77 are not limited to this example.

Markers M1 to M3 serving as the classification information and the relative values X1 to X3 are respectively displayed in the vicinities of the peaks in the unit respiratory waveforms 751 to 753. Incidentally, each of the markers M1 to M3 is hatched to represent the classified respiratory depth state of the subject P. Since all the relative values X1 to X3 corresponding to the unit respiratory waveforms 751 to 753 are not less than 50%, the respiratory depth state of the subject P corresponding to each of the unit respiratory waveforms 751 to 753 is “normal”. Therefore, the markers M1 to M3 are hatched (hatched with horizontal lines) to represent “normal”. The relative values X1 to X3 are displayed in the vicinities of left sides of the markers M1 to M3.

Next, processing performed by the controller 5 in the STEP07 after the STEP10 has been executed will be described in detail with reference to FIG. 6. Incidentally, FIG. 6 illustrates how the respiration of the subject P gradually becomes shallower due to the anesthetic. The controller 5 generates relative values X4 to X9 whenever unit respiratory waveforms 754 to 759 are acquired according to a principle similar to or the same as that used to generate the relative values X1 to X3. After generating the relative values X4 to X9, the controller 5 classifies respiratory depth state of the subject P corresponding to each of the unit respiratory waveforms 754 to 759 into one of the categories based on a corresponding one of the relative values X4 to X9, so as to generate a piece of classification information corresponding to the classified category. In the example illustrated in FIG. 6, the relative value X4 is not less than 50%, so that the controller 5 classifies the respiratory depth state of the subject P corresponding to the unit respiratory waveform 754 as “normal”. A marker M4 hatched (hatched with horizontal lines) to represent “normal” is located in the vicinity of a right side of the relative value X4. Since the relative values X5 to X7 are not less than 20% and not more than 49%, the controller 5 classifies the respiratory depth state of the subject P corresponding to each of the unit respiratory waveforms 755 to 757 as “cautionary”. Markers M5 to M7 hatched (hatched with vertical lines) to represent “cautionary” are located in the vicinities of right sides of the relative values X5 to X7. Since the relative values X8 to X9 are not more than 19%, the controller 5 classifies the respiratory depth state of the subject P corresponding to each of the unit respiratory waveforms 758 to 759 as “dangerous”. Markers M8 to M9 hatched (hatched with oblique lines) to indicate “dangerous” are located in the vicinities of right sides of the relative values X8 to X9. In this manner, the controller 5 continuously generates the respiratory depth information until the predetermined time elapses since the time instant at which the acquirer 2 started acquiring the post-surgery respiratory waveform data from the subject P.

Return to FIG. 2. STEP11 to STEP13 will be described. When, for example, the current time instant is 18:10, the controller 5 determines that the predetermined time (10 minutes) has elapsed since the time instant (18:00) at which the acquirer 2 started acquiring the post-surgery respiratory waveform data from the subject P (YES in the STEP08). In this case, the controller 5 generates aggregate information about the respiratory depth of the subject P in the predetermined time based on the pieces of the respiratory depth information continuously generated until 10 minutes have elapsed since the time instant at which the controller 5 started acquiring the respiratory waveform data from the subject P after the surgery (the STEP11).

As illustrated in FIG. 6, the controller 5 generates average respiratory depth information and cumulative number-of-times information as the aggregate information. Incidentally, the average respiratory depth information is an average value of relative magnitudes of second amplitudes to the first amplitude A1 in the predetermined time, such as an average value of relative magnitudes (the relative values X1 to X9) of second amplitudes A21 to A29 to the first amplitude A1 in the predetermined time. Moreover, the cumulative number-of-times information is information indicating a cumulative number of times for each category (i.e., dangerous, cautionary or normal) about the respiratory depth state.

In the presently disclosed embodiment, the average respiratory depth information is the average value of the relative values X1 to X9. Since the average value of the relative values X1 to X9 is 46%, the average value (46%) is the average respiratory depth information in the presently disclosed embodiment. However, the average respiratory depth information is not limited to the numerical value information such as the average value, but may be, for example, another information such as a level or characters representing the respiratory depth state (normal etc.). As for the cumulative number-of-times information, the controller 5 classifies the respiratory depth state of the subject P into one of the state categories for each of the unit respiratory waveforms based on the acquired respiratory waveform data and the respiratory reference waveform data, and counts up the number of times when the respiratory depth state has been classified into each of the state categories. Consequently, the controller 5 generates the cumulative number-of-times information. Incidentally, in the presently disclosed embodiment, discriminative signs S1 to S3 are used as discriminative signs representing the categories about the respiratory depth state. The discriminative signs S1 to S3 are hatched to represent the respiratory depth state of the subject P according to the criteria similar to or the same as those for the markers M1 to M9. A number displayed on each of right sides of the discriminative signs S1 to S3 indicates a cumulative number of times when the respiratory depth state has been classified into a corresponding one of the categories. That is, the controller 5 has classified and counted up the respiratory depth state of the subject P as “normal” 61 times, as “cautionary”45 times, and as “dangerous” 2 times based on the respiratory waveform data acquired for the 10 minutes since the respiratory waveform data started being acquired from the subject P after the surgery. The controller 5 generates the cumulative number-of-times information representing the counting results.

After generating the aggregate information, the controller 5 generates display data for displaying the respiratory waveform data, the respiratory depth information, and the aggregate information on the display 7 or on the display provided in the external apparatus 20 (the STEP12), as illustrated in FIG. 2. Incidentally, the controller 5 generates first display data in the STEP12 in a manner similar to or the same as in the STEP09. In the presently disclosed embodiment, the generated first display data are transmitted to the display 7.

When the first display data generated by the controller 5 are transmitted to the display 7, a display screen corresponding to the first display data is displayed on the display 7 in a manner similar to or the same as in the STEP10 (the STEP13).

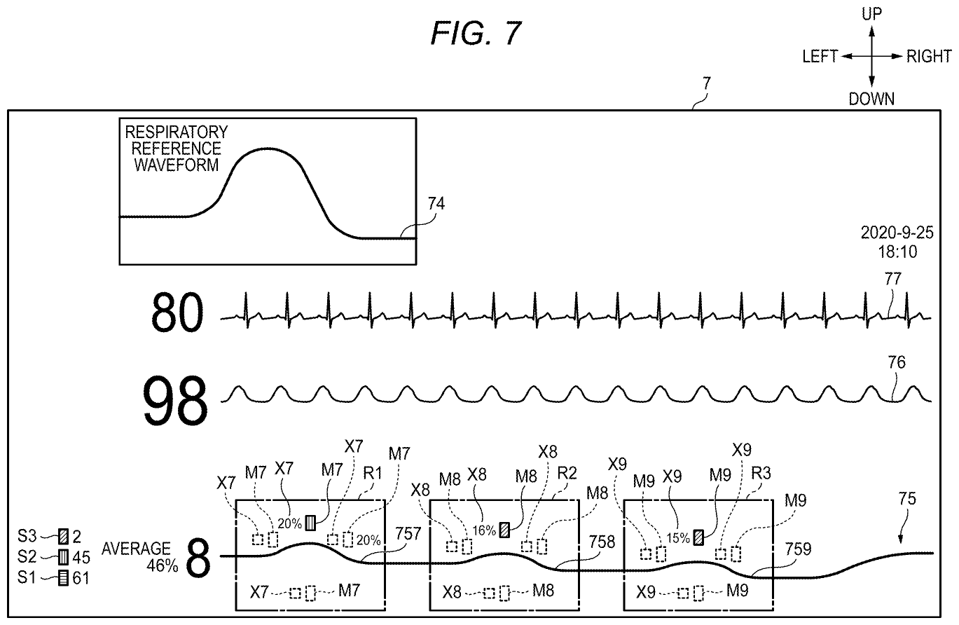

A display screen displayed on the display 7 at the time point 18:10 will be described here with reference to FIG. 7. However, description about parts similar to or the same as those of the display screen illustrated in FIG. 5 will be omitted for convenience of explanation. Incidentally, after the predetermined time (10 minutes) has elapsed, the aggregate information is updated based on pieces of respiratory depth information which were generated in the last 10 minutes. At the time point 18:10, the display screen illustrated in FIG. 7 is displayed on the display 7. The display screen illustrated in FIG. 7 differs from the display screen illustrated in FIG. 5 in that average respiratory depth information is displayed in the vicinity of a left side of a respiratory rate (“8” in FIG. 7), and discriminative signs S1 to S3 and pieces of cumulative number-of-times information are displayed in the vicinity of a left side of the average respiratory depth information.

The markers M7 to M9 (classification information) and the relative values X7 to X9 (numerical value information) are displayed in the vicinities of peaks in the unit respiratory waveforms 757 to 759. The markers M7 to M9 are hatched in the aforementioned manner. That is, since the relative value X7 corresponding to the unit respiratory waveform 757 is 20%, the marker M7 is hatched with vertical lines. In addition, since the relative value X8 corresponding to the unit respiratory waveform 758 is 16% and the relative value X9 corresponding to the unit respiratory waveform 759 is 15%, the markers M8 to M9 are hatched with oblique lines.

Next, a function of sending notification of the respiratory depth state of the subject P will be described. After determining that the respiratory depth state of the subject P is bad based on the respiratory depth information, the controller 5 determines that it is necessary to send notification of the respiratory depth state of the subject P. When, for example, the relative value calculated based on the respiratory depth information is not more than 49%, the controller 5 determines that the respiratory depth state of the examinee P is bad, and determines that it is necessary to send notification of the respiratory depth state of the subject P. In this case, the controller 5 generates a notification signal for notifying the medical worker of the respiratory depth state of the subject P, and transmits the generated notification signal to the notifier 8 or the external apparatus 20. On the other hand, when, for example, the relative value calculated based on the respiratory depth information is not less than 50%, the controller 5 determines that the respiratory depth state of the subject P is good. In this case, the controller 5 determines that it is not necessary to send notification of the respiratory depth state of the subject P, and therefore does not generate the notification signal.

After generating the notification signal, for example, the controller 5 transmits the generated notification signal to the notifier 8. Upon reception of the notification signal, the notifier 8 notifies the medical worker of the respiratory depth state of the subject P based on the received notification signal. When, for example, the medical worker around the information generating apparatus 1 is audibly notified of the respiratory depth state of the subject P, the notifier 8 outputs voice saying “The respiratory state of the subject P seems to be bad. Please check the condition of subject P.”. In addition, when, for example, the medical worker around the information generating apparatus 1 is visibly notified of the respiratory depth state of the subject P, the notifier 8 outputs character information indicating “The respiratory state of the subject P seems to be bad. Please check the condition of subject P.” on the display 7.

The functions which have been described so far can be realized by the memory 51 and the processor 52. A computer program for executing the aforementioned processing can be stored in the memory 51. The computer program may be stored in the memory 51 in advance, or may be downloaded from an external server through a communication network.

In addition, a computer-readable medium may be used in the presently disclosed embodiment. The computer-readable medium means any type of physical memory (RAM, ROM, or the like) that can store information or data readably by the processor 52. The computer-readable medium may store at least one command about processing to be executed by the processor. Incidentally, the term “computer-readable medium” includes a tangible article and excludes any carrier signal or any transitory signal (i.e. meaning a non-transitory medium). Examples of the non-transitory computer-readable medium include a magnetic recording medium (e.g. a flexible disk, a magnetic tape, or a hard disk drive), a magneto-optical recording medium (e.g. a magneto-optical disk), a CD-ROM (Read Only Memory), a CD-R, a CD-R/W, and a semiconductor memory (e.g. a mask ROM, a PROM (Programmable ROM), an EPROM (Erasable PROM), or a flash ROM).

By the way, the respiratory state of the subject is generally judged based on respiratory frequency and the respiratory depth. The respiratory frequency can be objectively determined from the respiratory rate, but the respiratory depth is subjectively judged by the medical worker who visually checks the respiratory waveform so that it is difficult for the medical worker to objectively judge the respiratory depth. To solve this problem, the present inventor came up with a solution that the respiratory depth could be objectively grasped by comparing the respiratory reference waveform and the respiratory waveform included in the respiratory waveform data acquired from the subject.

According to the aforementioned configuration, the respiratory reference waveform data serving as a reference for determining the respiratory depth of the subject P and the respiratory waveform data acquired from the subject are compared so that the respiratory depth information generated by the controller 5 is generated. That is, the respiratory depth information is information objectively representing the respiratory depth of the subject. Therefore, for example, the medical worker who visually checks the display 7 on which the respiratory depth information is displayed does not have to judge the respiratory depth based on the medical worker's personal experience etc. as in the background art but can judge the respiratory depth based on the respiratory depth information, which is an objective index. As a result, the medical worker can objectively grasp the respiratory depth of the subject P.

In addition, according to the aforementioned configuration, the respiratory depth information generated by the controller 5 represents the relative magnitudes of the second amplitudes A21 to A29 of the second respiratory waveform included in the respiratory waveform data to the first amplitude A1 of the first respiratory waveform included in the respiratory reference waveform data. Therefore, also in this case, the medical worker can judge the respiratory depth based on the respiratory depth information, which is an objective index so that the medical worker can objectively grasp the respiratory depth of the subject P.

In addition, according to the aforementioned configuration, the respiratory reference waveform data can be set based on the respiratory waveform data acquired in advance from the subject P. Since a respiratory waveform of one subject varies greatly from a respiratory waveform of another subject, it is desirable that the respiratory reference waveform data are set based on the respiratory waveform data acquired in advance from the target subject. Therefore, according to the aforementioned configuration, more accurate respiratory depth information can be generated.

In addition, according to the aforementioned configuration, the respiratory reference waveform data can be set based on the attribute information of the subject P. Therefore, for example, even in a case where there is no respiratory waveform data acquired in advance from the subject P, respiratory depth information can be generated by use of the respiratory reference waveform data set based on the attribute information of the subject P.

In addition, according to the aforementioned configuration, the attribute information includes age information, gender information, chronic disease information, pre-existing disease information etc. Therefore, even in the case where there is no respiratory waveform data acquired in advance from the subject P, for example, respiratory depth information is generated based on respiratory reference waveform data set based on statistical respiratory waveform data that have been acquired from a plurality of other persons with attributes close to the subject P. Therefore, even in the case where there is no respiratory waveform data acquired in advance from the subject P, relatively accurate respiratory depth information can be generated.

Moreover, according to the aforementioned configuration, the first amplitude A1 of the first respiratory waveform included in the respiratory reference waveform data and the second amplitudes A21 to A29 of the second respiratory waveform included in the respiratory waveform data are compared so that the respiratory depth information is generated. Since the first amplitude A1 and the second amplitudes A21 to A29 are determined by use of both the expiratory peak pressure and the inspiratory peak pressure, the controller 5 can easily generate the respiratory depth information in which the respiratory state of the subject P has been reflected more accurately.

Moreover, according to the aforementioned configuration, the respiratory depth information includes the classification information corresponding to the relative magnitudes of the second amplitudes A21 to A29 to the first amplitude A1. Therefore, the medical worker can objectively and easily grasp the respiratory depth state of the subject P by use of the classification information.

Moreover, according to the aforementioned configuration, the respiratory depth information includes the numerical value information representing the relative magnitudes of the second amplitudes A21 to A29 to the first amplitude A1. Therefore, since the numerical value information is used, the medical worker can know the respiratory depth state of the subject P also by use of the numerical value information so that the medical worker can grasp the respiratory depth state of the subject P more objectively and easily.

Moreover, according to the aforementioned configuration, the display data are generated so that pieces of the respiratory depth information are displayed in the vicinities of the unit respiratory waveforms 751 to 759. The medical worker who visually checks the display screen based on such display data can easily recognize which part of the respiratory waveform the respiratory depth information corresponds to. In particularly, when the display data are generated so that the pieces of the respiratory depth information are displayed in the vicinities of the peaks of the unit respiratory waveforms 751 to 759, the medical worker can more easily recognize which parts of the respiratory waveform the pieces of the respiratory depth information correspond to.

Moreover, according to the aforementioned configuration, the pieces of the respiratory depth information are generated to be displayed in association with the unit respiratory waveforms 751 to 759 respectively. The medical worker who visually checks the display screen based on such display data can grasp at a glance the respiratory depth of the subject in each breath in the predetermined time.

Moreover, according to the aforementioned configuration, the controller 5 generates the aggregate information about the respiratory depth of the subject P in the predetermined time, based on the pieces of the respiratory depth information continuously generated for the predetermined time. Thus, for example, the medical worker can easily grasp the outline of the respiratory depth of the subject P in the predetermined time by use of the aggregate information.

Moreover, according to the aforementioned configuration, the controller 5 generates either the first display data or the second display data as the display data. The first display data have the inspiratory pressure higher than the expiratory pressure in the respiratory waveform. The second display data have the expiratory pressure higher than the inspiratory pressure in the respiratory waveform. Therefore, the information generating apparatus 1 can provide the medical worker who visually checks the respiratory waveform a respiratory waveform display form satisfying the medical worker's preference.

Moreover, according to the aforementioned configuration, the information generating apparatus 1 is provided with the display 7 for displaying the respiratory waveform data and the respiratory depth information. Therefore, according to the information generating apparatus 1, the medical worker can visually check the respiratory waveform data and the respiratory depth information even in the case where there is no external apparatus 20 for displaying the respiratory waveform data and the respiratory depth information.

Moreover, according to the aforementioned configuration, the controller 5 generates a notification signal for sending notification of the respiratory depth state based on the respiratory depth information. Therefore, according to the information generating apparatus 1, in the case where, for example, the respiratory depth of the subject P is in a bad state, the medical worker can be notified of this state. As a result, the medical worker can immediately grasp the respiratory depth state of the subject P. Therefore, when the respiratory depth of the subject P is in the bad state, the medical worker can immediately perform appropriate treatment on the subject P.

### Modification of First Embodiment

Next, a modification of the first embodiment will be described with reference to FIG. 7. The presently disclosed modification differs from the first embodiment in that markers M7 to M9 (classification information) and relative values X7 to X9 (numerical value information) are displayed at ones of positions indicated by short dashed lines in FIG. 7. That is, the presently disclosed modification differs from the first embodiment in that pieces of respiratory depth information are displayed in the vicinities of unit respiratory waveforms. The vicinities of the unit respiratory waveforms are, for example, regions R1 to R3 enclosed by long dashed short dashed lines in FIG. 7. In other words, each of the vicinities of the unit respiratory waveforms is a position of a rising or falling edge of the unit respiratory waveform, a position above or under a peak of the unit respiratory waveform, or the like.

Also in the presently disclosed modification, each of the pieces of respiratory depth information is located in the vicinity of a corresponding one of the unit respiratory waveforms. Accordingly, a medical worker can easily recognize which part of a respiratory waveform the piece of respiratory depth information corresponds to.

### Second Embodiment

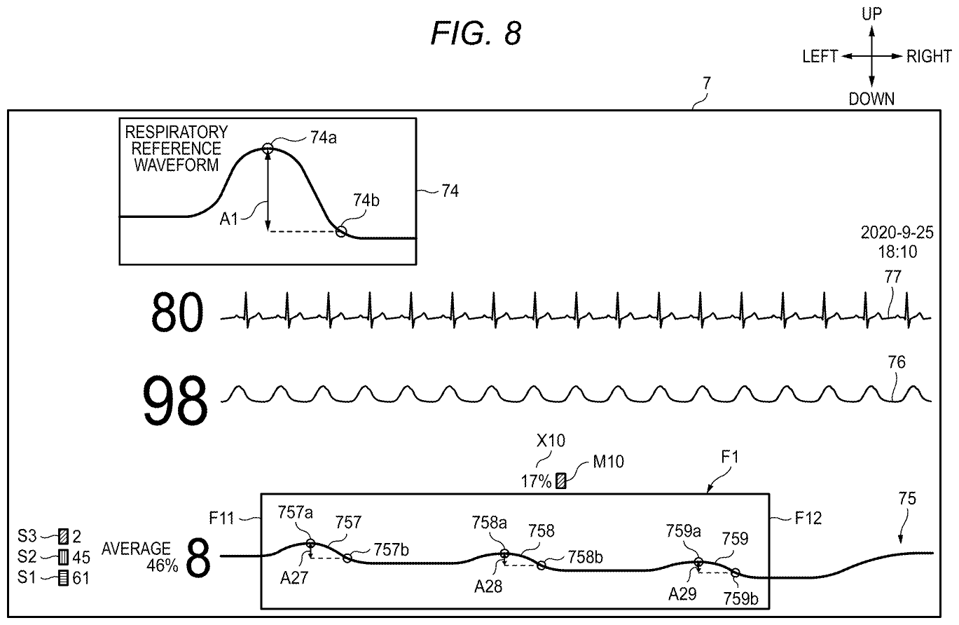

Next, a second embodiment will be described with reference to FIG. 8. The presently disclosed embodiment differs from the first embodiment in that respiratory depth information is generated from a unit of a data set including three consecutive unit respiratory waveforms. In other words, the respiratory depth information generated in the presently disclosed embodiment represents depths of three consecutive breaths taken by a subject P as a piece of information (e.g. an average value, a maximum value, a minimum value, or the like of relative values corresponding to the three consecutive breaths). Incidentally, the data set need only include two or more consecutive unit respiratory waveforms. The number of the unit respiratory waveforms included in the data set is not limited to three. In addition, in description of the presently disclosed embodiment, description about portions overlapping with the description in the first embodiment will be omitted appropriately.

For example, at a time point 18:10, a controller 5 calculates relative values X7 to X9 respectively according to a principle similar to or the same as that in the first embodiment. After calculating the relative values X7 to X9, the controller 5 calculates, for example, an average value X10 of the relative values X7 to X9, and classifies a respiratory depth state of the subject P based on the calculated average value X10 and classification criteria information. In the presently disclosed embodiment, the relative value X7 is 20%, the relative value X8 is 16%, and the relative value X9 is 15% (see FIG. 6). Accordingly, the average value X10 of the relative values X7 to X9 is 17%. Therefore, the controller 5 classifies the respiratory depth state of the subject P corresponding to a data set including unit respiratory waveforms 757 to 759 as “dangerous” and generates classification information corresponding to the category.

After classifying the respiratory depth state of the subject P corresponding to the data set including the unit respiratory waveforms 757 to 759, the controller 5 generates respiratory depth information representing the respiratory depth state and generates first display data based on the generated respiratory depth information. A display screen based on the first display data generated in the presently disclosed embodiment is a display screen illustrated in FIG. 8. As illustrated in FIG. 8, the unit respiratory waveforms 757 to 759 are enclosed by a rectangular frame line F1. A left side F11 of the frame line F1 passes through a place of a rising edge of the unit respiratory waveform 757, and a right side F12 of the frame line F1 passes through a place of a falling edge of the unit respiratory waveform 759.

The form of the frame line F1 can be changed according to the classified state. For example, the frame line F1 may be set to be thicker as the respiratory depth state is worse, or may be colored accordingly to the classified state. A marker M10 (classification information) hatched with oblique lines to represent “dangerous” and the average value X10 (numerical value information) are displayed on an outer side above the frame line F1. The relative value X10 is displayed in the vicinity of a left side of the marker M10. Incidentally, the marker M10 and the average value X10 may be displayed inside the frame line F1 or may be displayed in the vicinity of a left/right side of the frame line F1.

According to the aforementioned configuration, a medical worker who visually checks such a display screen can visually check the depths of the multiple breaths collectively as a piece of information.

The aforementioned embodiment is intended to make the presently disclosed subject matter easy to understand. However, the aforementioned embodiment is not intended to limit the presently disclosed subject matter. The presently disclosed subject matter may be modified and improved without departing from the spirit of the presently disclosed subject matter.

In the aforementioned embodiment, the controller 5 generates the respiratory depth information based on the relative values X1 to X9 calculated by comparing the first amplitude A1 and the second amplitudes A21 to A29. However, the presently disclosed embodiment is not limited thereto. For example, the controller 5 may generate the respiratory depth information based on similarity between the respiratory reference waveform 74 and the respiratory waveform 75, that is calculated by comparing the two waveforms.

In the aforementioned embodiment, the information generating apparatus 1 is not provided with a pressure sensor 10. However, the acquirer 2 of the information generating apparatus 1 may be provided with a pressure sensor having a configuration similar to or the same as the pressure sensor 10. In this case, the acquirer 2 detects respired air from at least one of the mouth and nose of the subject P, and acquires respiratory waveform data related to respiratory pressure of the subject P based on the detected respired air.

In the aforementioned embodiment, the information generating apparatus 1 is provided with the storage 4. However, the information generating apparatus 1 may be not provided with the storage 4. In this case, the memory 51 of the controller 5 functions as a storage.

In the aforementioned embodiment, the first value is the first amplitude A1 of the first respiratory waveform included in the respiratory reference waveform data, and the second value is the second amplitude A21 to A29 of the second respiratory waveform included in the respiratory waveform data. However, the presently disclosed embodiment is not limited thereto. For example, the first value may be maximum respiratory pressure in the entire respiratory reference waveform, and the second value may be maximum respiratory pressure in the entire respiratory waveform. In addition, the first value may be a value of average respiratory pressure in the first respiratory waveform until a predetermined time elapses since respiration starts, and the second value may be a value of average respiratory pressure in the second respiratory waveform until a predetermined time elapses since respiration starts. Further, the first value may be a value of maximum respiratory pressure in the first respiratory waveform until a predetermined time elapses since respiration starts, and the second value may be a value of maximum respiratory pressure in the second respiratory waveform until a predetermined time elapses since respiration starts. In addition, the first value may be a value of average respiratory pressure from a rising edge to a peak of the first respiratory waveform, and the second value may be a value of average respiratory pressure from a rising edge to a peak of the second respiratory waveform.

In the aforementioned embodiment, each marker is displayed as the classification information on the display 7. However, for example, character information indicating the respiratory depth state of the subject P may be displayed as the classification information. In addition, thickness of the unit respiratory waveform changed based on the respiratory depth state of the subject P may be used in place of the marker to represent the respiratory depth state of the subject P. In this case, the thickness of the unit respiratory waveform corresponds to the classification information.

In the aforementioned embodiment, each relative value is displayed as the numerical value information on the display 7. However, for example, one of levels 1 to 5 corresponding to the calculated relative value may be displayed alternatively. Incidentally, the number of the levels is not limited to five but may be two or more.

In the aforementioned embodiment, the marker and the relative value are displayed on the display 7. However, either the marker or the relative value may be not displayed on the display 7. In other words, the respiratory depth information may include only one of the classification information and the numerical value information.

In the aforementioned embodiment, the discriminative signs S1 to S3 and the cumulative number-of-times information are displayed on the display 7 at the time point 18:10. However, the discriminative signs S1 to S3 and the cumulative number-of-times information may be not displayed on the display 7.

In the aforementioned embodiment, different hatchings are added to the discriminative signs S1 to S3 and the markers M1 to M10 to distinguish the categories each representing the respiratory depth state from one another. However, the presently disclosed embodiment is not limited thereto. The categories may be distinguished from one another, for example, by giving different colors to the discriminative signs S1 to S3 and the markers M1 to M10, or by forming different shapes for the discriminative signs S1 to S3 and the markers M1 to M10. Moreover, the categories may be also distinguished from one another by giving different colors to the backgrounds of the respective unit respiratory waveforms or the respective unit respiratory waveforms based on the classification information.

The aforementioned embodiment has been described by use of an example in which physiological information including respiratory waveform data is acquired before and after surgery from a subject P administered an anesthetic during the surgery. However, the presently disclosed embodiment is not limited to this example. For example, the presently disclosed subject matter can be also applied to an example in which respiration of the subject P changes from tachypnea to hypopnea.

In the aforementioned embodiment, the respiratory depth state is classified as “normal”, “cautionary” or “dangerous”. However, it may be also classified as “level 1”, “level 2” or “level 3”.

In the aforementioned embodiment, the respiratory depth state is classified into the three “normal”, “cautionary” and “dangerous” states. However, it may be classified into two or four or more states.

In the aforementioned embodiment, the relative value is displayed in the vicinity of the left side of the marker. However, it may be displayed in the vicinity above the marker, the vicinity of the right side of the marker, or the like.

In the aforementioned embodiment, the respiratory reference waveform 74 is displayed on the display 7. However, the respiratory reference waveform 74 may be not displayed on the display 7. In other words, the controller 5 may generate display data that does not include the respiratory reference waveform data.

In the aforementioned embodiment, the controller 5 compares the respiratory waveform 75 with the respiratory reference waveform 74 so as to generate the respiratory depth information, and then generates the display data for displaying the respiratory waveform data and the respiratory depth information. However, for example, the controller 5 may be configured not to generate the respiratory depth information but to generate the display data for displaying the respiratory reference waveform data set in advance and the respiratory waveform data acquired by the acquirer 2. In this case, the controller 5 displays a display screen in which the respiratory reference waveform 74 and the respiratory waveform 75 are displayed on the display 7 or the display of the external apparatus 20 based on the generated display data. In addition, even when the controller 5 generates the respiratory depth information, the controller 5 may be configured to generate display data excluding the respiratory depth information in response to a setting operation of a user.

### REFERENCE SIGNS LIST

1: information generating apparatus, 2: acquirer; 3: operating unit; 4: storage; 5: controller; 6: output interface; 7: display; 8: notifier; 9: bus; 10: pressure sensor; 20: external apparatus; 51: memory; 52: processor

What is claimed is:

1. An information generating apparatus comprising:
an acquirer that is configured to acquire respiratory waveform data about respiratory pressure of a subject; and
a controller that is configured to compare the respiratory waveform data acquired by the acquirer with preset respiratory reference waveform data, so as to generate respiratory depth information representing respiratory depth of the respiratory waveform data with respect to the respiratory reference waveform data.

2. The information generating apparatus according to claim 1, wherein:
a second value representing respiratory pressure based on the respiratory waveform data is compared with a first value representing respiratory pressure based on the respiratory reference waveform data so that the respiratory depth information is generated.

3. The information generating apparatus according to claim 1, wherein:
the respiratory reference waveform data are set based on respiratory waveform data acquired in advance from the subject.

4. The information generating apparatus according to claim 1, wherein:
the respiratory reference waveform data are set based on attribute information about at least one attribute of the subject.

5. The information generating apparatus according to claim 4, wherein:
the attribute information includes at least one of age information, gender information, chronic disease information, and pre-existing disease information.

6. The information generating apparatus according to claim 1, wherein:
the respiratory reference waveform data include a first respiratory waveform having a first amplitude;
the respiratory waveform data include a second respiratory waveform having a second amplitude;
the first amplitude is determined based on expiratory peak pressure in the first respiratory waveform and inspiratory peak pressure in the first respiratory waveform;
the second amplitude is determined based on expiratory peak pressure in the second respiratory waveform and inspiratory peak pressure in the second respiratory waveform; and
the controller compares the first amplitude and the second amplitude so as to generate the respiratory depth information.

7. The information generating apparatus according to claim 6, wherein:
the respiratory depth information includes classification information according to relative magnitude of the second amplitude relative to the first amplitude.

8. The information generating apparatus according to claim 6, wherein:
the respiratory depth information includes numerical value information indicating relative magnitude of the second amplitude relative to the first amplitude.

9. The information generating apparatus according to claim 1, wherein:
the controller is configured to generate display data for displaying the respiratory waveform data and the respiratory depth information;
a respiratory waveform based on the respiratory waveform data includes at least one unit respiratory waveform corresponding to one breath taken by the subject; and
the display data are generated so that the respiratory depth information is displayed in the vicinity of the unit respiratory waveform.

10. The information generating apparatus according to claim 9, wherein:
the display data are generated so that the respiratory depth information is displayed in the vicinity of a peak of the unit respiratory waveform.

11. The information generating apparatus according to claim 9, wherein:
the respiratory waveform includes a plurality of the unit respiratory waveforms; and
the display data are generated so that the respiratory depth information is displayed in the vicinity of a peak in each of the unit respiratory waveforms.

12. The information generating apparatus according to claim 1, wherein:
the controller is configured to generate aggregate information about respiratory depths of the subject in a predetermined time based on pieces of the respiratory depth information continuously generated for the predetermined time.

13. The information generating apparatus according to claim 1, wherein:
the controller is configured to generate display data for displaying the respiratory waveform data and the respiratory depth information; and
the controller is configured to generate either first display data or second display data as the display data, the first display data having inspiratory pressure higher than expiratory pressure in a respiratory waveform based on the respiratory waveform data, the second display data having the expiratory pressure higher than the inspiratory pressure in the respiratory waveform.

14. The information generating apparatus according to claim 1, wherein:
the information generating apparatus includes a display; and
the controller is configured to generate display data for displaying the respiratory waveform data and the respiratory depth information on the display.

15. The information generating apparatus according to claim 1, wherein:
the controller is configured to generate a notification signal for sending notification of a respiratory depth state of the subject based on at least the respiratory depth information.

16. An information generating method that is attained by making an information generating apparatus execute:
a step of acquiring respiratory waveform data about respiratory pressure of a subject; and
a step of comparing the acquired respiratory waveform data with preset respiratory reference waveform data, so as to generate respiratory depth information representing respiratory depth of the respiratory waveform data with respect to the respiratory reference waveform data.

17. A non-transitory computer-readable medium on which a computer program has been recorded, the computer program making a computer realize:
a function of acquiring respiratory waveform data about respiratory pressure of a subject; and
a function of comparing the acquired respiratory waveform data with preset respiratory reference waveform data, so as to generate respiratory depth information representing respiratory depth of the respiratory waveform data with respect to the respiratory reference waveform data.
